SEQUENCE LISTING (1 sequence)

SEQ ID NO: 1 - protein, 20 aa, Artificial Sequence
  Synthetic peptide
VRPEHPAETEYDSLYPEDDL

(19) United States
(12) Patent Application Publication
(10) Pub. No.: US 2013/0012603 A1
(43) Pub. Date: Jan. 10, 2013

(54) METHODS FOR ASSESSING THE RISK OF CARDIOVASCULAR DISEASE

(76) Inventor: David H. Farrell, Tualatin, OR (US)

(21) Appl. No.: 13/634,719

(22) PCT Filed: Mar. 8, 2011
(86) PCT No.: PCT/US2011/027568

§ 371 Date: Sep. 13, 2012

Related U.S. Application Data
(60) Provisional application No. 61/314,134, filed on Mar. 15, 2010.

(51) Int. Cl. G01N 33/577; A61P 9/00; A61P 3/06; A61P 7/02; A61P 9/12; A61K 35/00; A61P 9/10

(52) U.S. Cl. 514/789; 435/7.92

(57) ABSTRACT

The studies described herein demonstrate that γ′ fibrinogen and total fibrinogen are independent risk factors for cardiovascular disease. Further described herein is the unexpected finding that an elevated concentration of γ′ fibrinogen in combination with an elevated concentration of total fibrinogen is a significantly better predictor of cardiovascular disease risk than either marker alone. Thus, provided herein are methods of detecting a subject having cardiovascular disease, or at increased risk of developing a cardiovascular disease, by measuring both the concentration of γ′ fibrinogen and the concentration of total fibrinogen in a sample obtained from the subject.

### CROSS REFERENCE TO RELATED APPLICATIONS

This application claims the benefit of U.S. Provisional Application No. 61/314,134, filed Mar. 15, 2010, which is herein incorporated by reference in its entirety.

### FIELD

This disclosure relates to methods of identifying a subject with increased risk of a cardiovascular disease, for example myocardial infarction or coronary artery disease, by measuring both the concentration of total fibrinogen and the concentration of γ′ fibrinogen in a sample obtained from the subject.

### BACKGROUND

The association between fibrinogen and cardiovascular disease (CVD) is well established by many epidemiologic studies, which show that fibrinogen is a significant risk factor for CVD (Danesh et al., JAMA 294:1799-1809, 2005). In addition, several lines of evidence point to a role for fibrin in acute thrombosis, including the ability of fibrinolytic agents such as tissue-type plasminogen activator and streptokinase to re-establish vascular patency after myocardial or cerebral infarction (Collen and Lijnen, Thromb Haemost 93:627-630, 2005); the first order relationship between fibrinolytic rates and initial fibrinogen concentration (Falls and Farrell, J Biol Chem 272:14251-14256, 1997; Kim et al., J Thromb Haemost 5:1250-1256, 2007); the effect of the factor XIII polymorphism Val34Leu in clot formation and its cardioprotective consequences (de Lange et al., Arterioscler Thromb Vasc Biol 26:1914-1919, 2006); and the decreased infarct size in fibrinogen knockout mice (Petzelbauer et al., Nature Med 11:298-304, 2005). However, possible mechanisms by which fibrinogen may contribute to CVD continue to be a subject of controversy. Elevated levels of fibrinogen have been proposed to promote fibrin formation, increase platelet aggregation, increase plasma viscosity, or simply reflect an inflammatory state (Stec et al., Circulation 102:1634-1638, 2000), but a mechanistic role for elevated fibrinogen in CVD remains elusive.

Fibrinogen is a heterogeneous mixture of isoforms with varying relative proportions. Alternative mRNA processing and post-translational modifications give rise to several different fibrinogen isoforms with widely varying characteristics (Lord, Curr Opin Hematol 14:236-241, 2007).

Fibrinogen consists of two copies each of three polypeptide chains, the Aα, Bβ, and γ chains, assembled with the stoichiometry (AαBβγ)(AαBβγ). There are alternatively processed mRNAs for both the Aα and γ′ chains. In particular, the γ chain gene gives rise to two different types of transcripts (Chung and Davie, Biochemistry 23:4232-4236, 1984; Fornace et al., J Biol Chem 259:12826-12830, 1984), one that encodes the more abundant γA chain and one that encodes the γ chain (FIG. 6). The γ′ chain makes up approximately 5% of the total γ chains (Mosesson et al., J Biol Chem 247:5223-5227, 1972), and can be assembled with either a γA chain or a γ′ chain. Fibrinogen that contains γ′ chains has either the stoichiometry (AαBβγA)(AαBβγ′) or (AαBβγ′)(AαBβγ′), and is often referred to as γ/γ′ fibrinogen or γ′/γ′ fibrinogen, respectively. Approximately 95% of fibrinogen molecules containing γ′ chains are γA/γ′ fibrinogen, whereas approximately 5% are γ′/γ′ fibrinogen.

γ′ fibrinogen has very different properties than the more common isoform that contains only γA chains. γ′ fibrinogen is a carrier protein for the unactivated zymogen form of factor XIII (Siebenlist et al., Biochemistry 35:10448-10453, 1996; Moaddel et al., Biochemistry 39:6698-6705, 2000). During coagulation, factor XIIIa is released by thrombin activation. The γ′ chain can also serve as a high affinity thrombin binding site during coagulation (Meh et al., J Biol Chem 271:23121-23125, 1996; Lovely et al., J Thromb Haemost 1:124-131, 2003; Pospisil et al., J Biol Chem 278:21584-21591, 2003; Sabo et al., Biochemistry 45:7434-7445, 2006; Pineda et al., Biophysical Chem 125:556-559, 2007). Most likely as a result of these processes, γ′ fibrinogen forms clots that are resistant to fibrinolysis (Falls and Farrell, J Biol Chem 272:14251-14256, 1997; Collet et al., Arterioscler Thromb Vasc Biol 24:382-386, 2004; Siebenlist et al., Blood 106:2730-2736, 2005) and has been associated with CVD in small case/control studies (Drouet et al., Blood Coagul Fibrinolysis 10 Suppl 1:S35-S39, 1999; Lovely et al., Thromb Haemost 88:26-31, 2002; Mannila et al., J Thromb Haemost 5:766-773, 2007; Cheung et al., Stroke 39:1033-1035, 2008).

### SUMMARY

It is disclosed herein that γ′ fibrinogen and total fibrinogen are independent risk factors for cardiovascular disease. Further described herein is the unexpected finding that an elevated concentration of γ′ fibrinogen in combination with an elevated concentration of total fibrinogen is a significantly better predictor of cardiovascular disease risk than either marker alone.

Thus, provided herein are methods of detecting a subject having a cardiovascular disease, or at increased risk of developing a cardiovascular disease. In some embodiments, the method includes measuring the concentration of γ′ fibrinogen and the concentration of total fibrinogen in a sample, such as a blood or plasma sample, obtained from the subject; comparing the measured concentration of γ′ fibrinogen and total fibrinogen to control ranges of γ′ fibrinogen and total fibrinogen in a population; and determining which tertile the measured concentrations of γ′ fibrinogen and total fibrinogen fall into compared to the concentrations of γ′ fibrinogen and total fibrinogen in the population. The subject is identified as having a cardiovascular disease, or at increased risk of developing a cardiovascular disease, if the measured concentration of both γ′ fibrinogen and total fibrinogen are in the top tertile.

In other embodiments, the method includes measuring the concentration of γ′ fibrinogen and the concentration of total fibrinogen in a plasma sample obtained from the subject, wherein a γ′ fibrinogen concentration of at least 0.20 mg/ml, and a total fibrinogen concentration of at least 3.0 mg/ml identifies the subject as having a cardiovascular disease, or at increased risk of developing a cardiovascular disease.

The foregoing and other features and advantages will become more apparent from the following detailed description of several embodiments, which proceeds with reference to the accompanying figures.

### BRIEF DESCRIPTION OF THE FIGURES

FIG. 1 is a graph showing analytical specificity of 2.G2.H9 toward γ′ fibrinogen. The analytical specificity of anti-γ′ fibrinogen monoclonal antibody 2.G2.H9 and its ability to differentiate γ′ fibrinogen from fibrinogen lacking γ′ chains was determined by incubating the indicated dilutions of the antibody with γA/γA or γ/γ′ fibrinogen in microtiter wells. Goat anti-mouse IgG/HRP conjugate was added for detection, followed by O-phenylenediamine, and the absorbance was quantitated at 450 nm.

FIG. 2 is a graph showing curve fitting for the γ′ fibrinogen ELISA. A γ′ fibrinogen standard curve was generated using the indicated concentrations of purified γ′ fibrinogen reconstituted in heat-defibrinated plasma. Capture antibody was anti-γ′ fibrinogen monoclonal antibody 2.G2.H9, and detection antibody was a commercial sheep anti-human fibrinogen/HRP conjugate. TMB substrate absorbance was quantitated at 450 nm. The resulting points were fit either to a linear, logarithmic or second-degree polynomial curve fit.

FIG. 3 is a graph showing the limit of quantification for the γ′ fibrinogen ELISA. The limit of quantification of the assay for measurement of γ′ fibrinogen concentrations in plasma was determined using eight separate pools of patient plasma. The limit of quantification for the assay was 0.10 g/L, defined as that concentration of γ′ fibrinogen giving a within-run CV of 20% or greater.

FIG. 4 is a graph showing γ′ fibrinogen concentrations in healthy individuals from the Framingham Offspring Study. γ′ fibrinogen was measured in 2,879 participants from the Framingham 1 Offspring Study with no previous history of cardiovascular disease. The reference interval of γ′ fibrinogen varied nearly 40-fold, from a low of 0.037 g/L to a high of 1.443 g/L. The 2.5^{th} and 97.5^{th} percentile limits for γ′ fibrinogen were 0.088 to 0.551 g/L. The median concentration was 0.234 g/L and the mean concentration was 0.255±0.119 g/L (±SD).

FIG. 5 is a graph showing receiver-operating characteristic (ROC) curve for γ′ fibrinogen in cardiovascular disease (CAD) patients. γ′ fibrinogen concentrations were measured as described in a prior study (Lovely et al., Thromb Haemost 88:26-31, 2002) in 133 patients referred for elective diagnostic cardiac catheterization. The receiver-operating characteristic curve of γ′ fibrinogen concentrations in CAD cases and controls showed an area under the curve of 0.76. A maximum diagnostic accuracy of 0.78 was found at a decision threshold of 0.30 g/L.

FIG. 6 is a schematic showing alternative splicing of the γ′ chain gene. It is hypothesized that competition between spliceosome cleavage of intron 9, which removes the intron and generates the γA mRNA, versus polyadenylation within intron 9 at the AAUAAA site that cleaves off the 3′ end of the pre-mRNA to generate the γ′ mRNA, regulates the ratio of γA to γ′ mRNA.

FIG. 7 is a three-dimensional graph showing the adjusted odds ratios for coronary heart disease (excluding angina) comparing the highest tertiles of γ′ fibrinogen and total fibrinogen with the lowest tertiles. Odds ratios were adjusted for sex, age, BMI, smoking, diabetes, fasting blood glucose, systolic blood pressure, total cholesterol, HDL cholesterol, and triglycerides.

FIG. 8 is a plot showing the results of a genome-wide association analysis for γ′ fibrinogen levels. -Log P values are shown across 22 autosomal and sex chromosomes. Multivariate-adjusted natural log-transformed γ′ fibrinogen levels include the covariates sex, age, BMI, systolic blood pressure, fasting blood glucose, diabetes mellitus, smoking, total cholesterol, HDL cholesterol, and triglycerides, plus principal components that account for potential population admixture. Each chromosome is indicated by alternating shading. The dashed gray horizontal line corresponds to the P value threshold of 5.0×10⁻⁸.

### SEQUENCE LISTING

The amino acid sequence listed in the accompanying sequence listing is shown using standard three letter code for amino acids, as defined in 37 C.F.R. 1.822. In the accompanying sequence listing:

SEQ ID NO: 1 is a peptide corresponding to the carboxyl terminal 20 amino acids of human γ′ fibrinogen.

### DETAILED DESCRIPTION

### I. Abbreviations

BMI body mass index

BSA bovine serum albumin

CAD coronary artery disease

CRP C-reactive protein

CVD cardiovascular disease

ELISA enzyme-linked immunosorbent assay

HDL high density lipoprotein

PBS phosphate-buffered saline

ROC receiver-operating characteristic

SNP single nucleotide polymorphism

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

To facilitate review of the various embodiments of the invention, the following explanations of specific terms are provided:

Antibody:

A polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology, Paul ed., 3d ed., 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554, 1990).

For preparation of antibodies, for example, recombinant, monoclonal, or polyclonal antibodies, many techniques known in the art can be used (see, e.g., Kohler & Milstein, Nature 256:495-497, 1975); Kozbor et al., Immunology Today 4:72 (1983); Cole et al., pp. 77-96 in Monoclonal Antibodies and Cancer Therapy, Alan R. Liss, Inc., 1985); Coligan, Current Protocols in Immunology (1991); Harlow & Lane, Antibodies, A Laboratory Manual (1988); and Goding, Monoclonal Antibodies: Principles and Practice, 2d ed., 1986). The genes encoding the heavy and light chains of an antibody of interest can be cloned from a cell, for example, the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology, 3.sup.rd ed. 1997). Techniques for the production of single chain antibodies or recombinant antibodies (described in, e.g., U.S. Pat. Nos. 4,946,778 and 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; and 5,661,016; Marks et al., Bio/Technology 10:779-783, 1992); Lonberg et al., Nature 368:856-859, 1994; Morrison, Nature 368:812-813, 1994); Fishwild et al., Nature Biotechnology 14:845-851, 1996; Neuberger, Nature Biotechnology 14:826, 1996; and Lonberg & Huszar, Intern. Rev. Immunol. 13:65-93, 1995).

Alternatively, phage display technology can be used to identify antibodies and heteromeric Fab fragments that specifically bind to selected antigens (see, e.g., McCafferty et al., Nature 348:552-554, 1990; Marks et al., Biotechnology 10:779-783, 1992). Antibodies can also be made bispecific (able to recognize two different antigen; see, e.g., PCT Publication No. WO 93/08829; Traunecker et al., EMBO J. 10:3655-3659, 1991; and Suresh et al., Methods in Enzymology 121:210, 1986). Antibodies can also be heteroconjugates, e.g., two covalently joined antibodies, or immunotoxins (see, e.g., U.S. Pat. No. 4,676,980; PCT Publication Nos. WO 91/00360 and WO 92/200373; and EP 03089).

In one embodiment, the antibody is conjugated to an effector moiety. The effector moiety can be any number of molecules, including labeling moieties such as radioactive labels or fluorescent labels for use in diagnostic assays.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

Antibodies for use in the methods of this disclosure can be monoclonal or polyclonal. Merely by way of example, monoclonal antibodies can be prepared from murine hybridomas according to the classical method of Kohler and Milstein (Nature 256:495-97, 1975) or derivative methods thereof. Detailed procedures for monoclonal antibody production are described in Harlow and Lane, Using Antibodies: A Laboratory Manual, CSHL, New York, 1999.

Cardiovascular Disease (CVD):

A group of diseases that includes, but is not limited to, atherosclerosis, coronary artery disease (CAD), angina pectoris (commonly known as “angina”), thrombosis, ischemic heart disease, coronary insufficiency, peripheral vascular disease, myocardial infarction, cerebrovascular disease (such as stroke), transient ischemic attack, arteriolosclerosis, small vessel disease, elevated cholesterol, intermittent claudication or hypertension.

Fibrinogen:

Fibrinogen is a dimeric glycoprotein complex of approximately 340,000 Da that consists of a mixture of isoforms that differ in both primary structure and post-translational modifications (Lord, Curr Opin Hematol 14:236-241, 2007). Fibrinogen consists of two copies each of three polypeptide chains, the Aα, Bβ, and γ chains, assembled with the stoichiometry (AαBβγ)(AαBβγ). “A” and “B” represent two small amino terminal peptides, known as fibrinopeptide A and fibrinopeptide B, respectively. There are alternatively processed mRNAs for both the Aα and γchains. In particular, the γ chain gene gives rise to two different types of transcripts (Chung and Davie, Biochemistry 23:4232-4236, 1984; Fornace et al., J Biol Chem 259:12826-12830, 1984), one that encodes the more abundant γA chain and one that encodes the γ′ chain. In human plasma, about 90% of the fibrinogen present is γA/γA-fibrinogen, and the remaining 10% is γA/γ′-fibrinogen. The γ′ chain makes up approximately 5% of the total γ chains (Mosesson et al., J Biol Chem 247:5223-5227, 1972), and can be assembled with either a γA chain or a γ′ chain. Fibrinogen that contains a γ′ chain has either the stoichiometry (AαBβγA)(AαBβγ′) or (AαBβγ′)(AαBβγ′), and is often referred to as γA/γ′ fibrinogen or γ′/γ′ fibrinogen, respectively. Approximately 95% of fibrinogen molecules containing a γ′ chain are γA/γ′ fibrinogen, whereas approximately 5% are γ′/γ′ fibrinogen.

γ′ Fibrinogen:

As used herein, γ′ fibrinogen refers fibrinogen containing at least one γ′ chain, including both γA/γ′ and γ′/γ′ fibrinogen.

Isolated:

An “isolated” biological component (such as a nucleic acid molecule, protein, or cell) has been substantially separated or purified away from other biological components in an organism, in which the component naturally occurs, such as a protein. In one example, the biological component is serum or plasma which has been isolated from a subject, for example from a blood sample obtained from the subject.

Label:

A composition detectable by (for instance) spectroscopic, photochemical, biochemical, immunochemical, or chemical means. Typical labels include fluorescent proteins or protein tags, fluorophores, radioactive isotopes (including for instance ³²P), ligands, biotin, digoxigenin, chemiluminescent agents, electron-dense reagents (such as metal sols and colloids), and enzymes (e.g., for use in an ELISA), haptens, and proteins or peptides (such as epitope tags) for which antisera or monoclonal antibodies are available. Methods for labeling and guidance in the choice of labels useful for various purposes are discussed, e.g., in Sambrook et al., in Molecular Cloning: A Laboratory Manual, Cold Spring Harbor Laboratory Press (1989) and Ausubel et al., in Current Protocols in Molecular Biology, John Wiley & Sons, New York (1998). A label often provides or generates a measurable signal, such as radioactivity, fluorescent light or enzyme activity, which can be used to detect and/or quantitate the amount of labeled molecule.

Mammal:

This term includes both human and non-human mammals. Similarly, the term subject includes both human and veterinary subjects, for example, humans, non-human primates, mice, rats, dogs, cats, horses, and cows.

Monoclonal Antibody 2.G2.H9:

A monoclonal antibody that specifically recognizes the γ′ chain of fibrinogen. 2.G2.H9 (described in U.S. Patent Application Publication No. 2003/0003515, incorporated herein by reference) was raised against a synthetic peptide corresponding to the 20 amino-terminal residues of γ′ fibrinogen. This antibody does not recognize γA fibrinogen. 2.G2.H9 is commercially available, such as from Santa Cruz Biotechnology and Millipore.

Myocardial Infarction:

Interruption of the blood supply to a part of the heart. Myocardial infarctions (also known as heart attacks) are commonly caused by an occlusion (blockage) of a coronary artery, such as an occlusion caused by an atherosclerotic plaque.

Odds Ratio:

The ratio of the odds of an event occurring in one group to the odds of it occurring in another group, or to a sample-based estimate of that ratio. These groups might be men and women, an experimental group and a control group, or any other dichotomous classification. If the probabilities of the event in each of the groups are p1 (first group) and p2 (second group), then the odds ratio is:

{p_{—}1/(1−p_{—}1)/(p_{—}2/(1−p_{—}2)}={p_{—}1/q_{—}1)/(p_{—}2/q_{—}2}={p_{—}1q_{—}2}/{p_{—}2q_{—}1},

where qx=1−px. An odds ratio of 1 indicates that the condition or event under study is equally likely to occur in both groups. An odds ratio greater than 1 indicates that the condition or event is more likely to occur in the first group. And an odds ratio less than 1 indicates that the condition or event is less likely to occur in the first group. The odds ratio must be greater than or equal to zero if it is defined. It is undefined if p2q1 equals zero.

For example, suppose that in a sample of 100 men, 90 have had blood tests in the previous week, while in a sample of 100 women only 20 have had blood tests in the same period. The odds of a man having a blood test are 90 to 10, or 9:1, while the odds of a woman having a blood test are only 20 to 80, or 1:4=0.25:1. The odds ratio is thus 9/0.25, or 36, showing that men are much more likely to have a blood test than women. Using the above formula for the calculation yields the same result:

{0.9/0.1)/(0.2/0.8}=(0.9×0.8)/(0.1×0.2)=0.72/0.02=36.

The above example also shows how odds ratios are sometimes sensitive in stating relative positions: in this sample men are 90/20=4.5 times more likely to have blood tests than women, but have 36 times the odds.

Plasma:

The yellow liquid component of blood that makes up about 55% of the total blood volume. Plasma is primarily made up of water (about 93%) and contains dissolved proteins, glucose, clotting factors (including fibrinogen), mineral ions, hormones and carbon dioxide (plasma does not contain blood cells). Plasma is prepared from a blood sample by centrifugation to pellet blood cells. In contrast, serum is blood plasma without fibrinogen or other clotting factors.

Population:

In the context of the present disclosure, population refers to any selected group of individuals, such as individuals that live in a particular geographic region, country or state. In some cases, the population is a group of subjects, such as a group of subjects that participated in a clinical study. In particular examples, the population is the Framingham Offspring Cohort.

Purified:

The term “purified” does not require absolute purity; rather, it is intended as a relative term. For example, a purified serum or plasma sample is one in which the serum or plasma has been substantially separated from the other components present in a blood sample.

Sample:

A biological specimen containing genomic DNA, RNA (including mRNA), protein, or combinations thereof, obtained from a subject. In particular examples, a sample is or includes a blood sample taken from a subject. Elements contained within the sample may be isolated, for example, plasma may be isolated from a blood sample.

Stroke (Ischemic Stroke):

The rapidly developing loss of brain function due to a disturbance in the blood supply to the brain. There are two categories of stroke, “ischemic stroke” and “hemorrhagic stroke.” Ischemic stroke refers to a condition that occurs when an artery to or in the brain is partially or completely blocked such that the oxygen demand of the tissue exceeds the oxygen supplied. Ischemic stroke is also referred to as “cerebral ischemia.” Deprived of oxygen and other nutrients following an ischemic stroke, the brain suffers damage as a result of the stroke.

Ischemic stroke is by far the most common kind of stroke, accounting for about 80% of all strokes.

Ischemic stroke can be caused by several different kinds of diseases. The most common problem is narrowing of the arteries in the neck or head. This is most often caused by atherosclerosis, or gradual cholesterol deposition. If the arteries become too narrow, blood cells may collect in them and form blood clots (thrombi). These blood clots can block the artery where they are formed (thrombosis), or can dislodge and become trapped in arteries closer to the brain (embolism). Another cause of stroke is blood clots in the heart, which can occur as a result of irregular heartbeat (for example, atrial fibrillation), myocardial infarction, or abnormalities of the heart valves, such as aortic valvular insufficiency.

Subject:

Living multi-cellular vertebrate organisms, a category that includes both human and non-human mammals.

Subject Having Increased Risk of a Disease or Condition:

A subject capable of, prone to, or predisposed to developing a disease or condition. It is understood that a subject already having or showing symptoms of a disease or condition is considered “susceptible” since they have already developed the disease or condition. A subject having increased risk of cardiovascular disease is a subject capable of, prone to, or predisposed to developing cardiovascular disease, including myocardial infarction.

Tertile:

One of three groups of an ordered distribution that is divided into three parts. For example, a data set (such as the concentration of fibrinogen in each individual of a population group) can be divided into three groups such that the bottom (or lowest) tertile represents the data points at the lowest end of the distribution (the lowest concentrations of fibrinogen), the top (or highest) tertile represents the data points at the highest end of the distribution (the highest concentrations of fibrinogen) and the middle tertile represents all data points between the top tertile and bottom tertile (the middle concentrations of fibrinogen). In particular non-limiting examples, the tertiles for/fibrinogen are: <0.198 mg/ml (bottom tertile), 0.198-0.286 mg/ml (middle tertile), and >0.286 mg/ml (top tertile); and the tertiles for total fibrinogen are: <3.09 mg/ml (bottom tertile), 3.09-3.68 mg/ml (middle tertile), and >3.68 mg/ml (top tertile).

Under Conditions Sufficient for:

A phrase used to describe any environment or set of conditions that permits the desired activity or outcome.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Introduction

The association between total fibrinogen concentration and cardiovascular disease (CVD) is well established by many epidemiologic studies, which show that fibrinogen is a significant risk factor for CVD (Danesh et al., JAMA 294:1799-1809, 2005). In addition, recent studies suggest that γ′ fibrinogen is a risk factor for CVD (Farrell, Curr Opin Hematol 11:151-155, 2004; Uitte de Willige et al., Blood 114:3994-4001, 2009; Cheung et al., Stroke 39:1033-1035, 2008). An association has been found between γ′ fibrinogen concentrations and prevalent coronary artery disease (CAD) (Lovely et al., Thromb Haemost 88:26-31, 2002), myocardial infarction (Mannila et al., J Thromb Haemost 5:766-773, 2007) and stroke (Cheung et al., Stroke 39:1033-1035, 2008). An earlier study found an association between the ratio of γ′ fibrinogen to total fibrinogen and myocardial infarction (Drouet et al., Blood Coagul Fibrinolysis 10 Suppl 1:S35-S39, 1999). However, prior to the present disclosure, it was believed in the art that γ′ fibrinogen and total fibrinogen were not independent biomarkers of CVD.

Moreover, studies of disease associations with γ′ fibrinogen, a newly-emerging risk factor for cardiovascular disease, have been hampered by the lack of a standardized and well characterized assay. This biomarker is associated with CVD in small case/control studies, but larger community-based studies on the association of γ′ fibrinogen with CVD or CVD risk factors are lacking. Thus there remains a need in the art for an assay which can reliably provide a clinical utility for assessment of an individual's risk of having CVD and CAD based on a correlation between γ′ fibrinogen and these conditions.

### IV. Overview of Several Embodiments

Disclosed herein is the finding that γ′ fibrinogen and total fibrinogen are independent risk factors for cardiovascular disease. Further disclosed herein is the unexpected finding that an elevated concentration of γ′ fibrinogen in combination with an elevated concentration of total fibrinogen is a significantly better predictor of cardiovascular disease risk than either marker alone.

Accordingly, provided herein are methods of detecting a subject having a cardiovascular disease, or at increased risk of developing a cardiovascular disease. In some embodiments, the method includes measuring the concentration of γ′ fibrinogen and the concentration of total fibrinogen in a blood sample obtained from the subject; comparing the measured concentration of γ′ fibrinogen and total fibrinogen to control ranges of γ′ fibrinogen and total fibrinogen in a population; and determining which tertile the measured concentrations of γ′ fibrinogen and total fibrinogen fall into compared to the concentrations of γ′ fibrinogen and total fibrinogen in the population. The subject is diagnosed with a cardiovascular disease, or at increased risk of developing a cardiovascular disease, if the measured concentration of both γ′ fibrinogen and total fibrinogen are in the top tertile.

In some examples, the top tertile for γ′ fibrinogen is a concentration of at least 0.20, at least 0.22, at least 0.24, at least 0.26, at least 0.28, at least 0.30, at least 0.32, at least 0.34, at least 0.36, at least 0.38, or at least 0.40 mg/ml.

In some examples, the top tertile for total fibrinogen is a concentration of at least 3.0, at least 3.2, at least 3.4, at least 3.6, at least 3.8, at least 4.0, at least 4.2, at least 4.4, at least 4.6, at least 4.8 or at least 5.0 mg/ml.

In one particular non-limiting example, the top tertile for γ′ fibrinogen is a concentration of at least 0.286 mg/ml and the top tertile for total fibrinogen is a concentration of at least 3.68 mg/ml.

In some embodiments, γ′ fibrinogen and total fibrinogen are measured in plasma isolated from the blood sample.

In one non-limiting example, the method includes measuring the concentration of γ′ fibrinogen and the concentration of total fibrinogen in a plasma sample obtained from the subject; comparing the measured concentration of γ′ fibrinogen and total fibrinogen to control ranges of plasma γ′ fibrinogen and total fibrinogen in a population; and determining which tertile the measured concentrations of γ′ fibrinogen and total fibrinogen fall into compared to the concentrations of γ′ fibrinogen and total fibrinogen in the population. The subject is identified as having a cardiovascular disease, or at increased risk of developing a cardiovascular disease, if the measured concentration of both γ′ fibrinogen and total fibrinogen are in the top tertile.

The population used for comparison can be any selected group of individuals, such as individuals that live in a particular geographic region, country or state. In some embodiments, the population is a group of subjects, such as a group of subjects that participated in a clinical study. In particular examples, the population is the Framingham Offspring Cohort. One of skill in the art will understand that the population used for comparison can vary. One of skill in the art is capable of selecting an appropriate population, such as a population in the same geographic region as the subject to be tested.

In other embodiments, the method of detecting a subject having a cardiovascular disease, or at increased risk of developing a cardiovascular disease, includes measuring the concentration of γ′ fibrinogen and the concentration of total fibrinogen in a plasma sample obtained from the subject, wherein a γ′ fibrinogen concentration of at least 0.20 mg/ml, and a total fibrinogen concentration of at least 3.0 mg/ml diagnoses the subject with a cardiovascular disease, or at increased risk of developing a cardiovascular disease. In some examples, a γ′ fibrinogen concentration of at least 0.20, at least 0.22, at least 0.24, at least 0.26, at least 0.28, at least 0.30, at least 0.32, at least 0.34, at least 0.36, at least 0.38, or at least 0.40 mg/ml, and a total fibrinogen concentration of at least 3.0, at least 3.2, at least 3.4, at least 3.6, at least 3.8, at least 4.0, at least 4.2, at least 4.4, at least 4.6, at least 4.8 or at least 5.0 mg/ml diagnoses the subject with a cardiovascular disease, or at increased risk of developing a cardiovascular disease. In one particular non-limiting example, a γ′ fibrinogen concentration of at least 0.286 mg/ml and a total fibrinogen concentration of at least 3.68 mg/ml diagnoses the subject with a cardiovascular disease, or at increased risk of developing a cardiovascular disease.

In some embodiments of the methods disclosed herein, the cardiovascular disease is atherosclerosis, coronary artery disease, angina, thrombosis, ischemic heart disease, transient ischemic attack, coronary insufficiency, peripheral vascular disease, myocardial infarction, cerebrovascular disease, intermittent claudication, hypertension or elevated cholesterol.

In particular examples, the cardiovascular disease is myocardial infarction or coronary artery disease.

In some embodiments, the methods further include obtaining the plasma sample from the subject.

Methods of measuring the concentration of total fibrinogen and γ′ fibrinogen are known in the art and any suitable procedure may be used in the disclosed methods. Exemplary procedures for measuring total fibrinogen and γ′ fibrinogen are described below. In some embodiments, measuring the concentration of γ′ fibrinogen includes contacting the sample with an antibody that specifically binds to γ′ fibrinogen, such as a monoclonal antibody specific for γ′ fibrinogen. In particular examples, the monoclonal antibody is 2.G2.H9.

In some embodiments, total fibrinogen concentration is measured in a sample based on the thrombin clotting time (e.g., using the Clauss method (Acta Haematol 17:237-246, 1957) or variations thereof). In other embodiments, total fibrinogen is measured by evaluating prothrombin time (PT-Fg), using a clottable protein assay or with an immunologic assay, such as an ELISA or immunoprecipitation test.

Suitable procedures for measuring total fibrinogen and γ′ fibrinogen concentrations can be determined by one of skill in the art.

In some embodiments, the method further includes reducing cardiovascular disease risk in the subject identified as having a cardiovascular disease or at increased risk of developing a cardiovascular disease.

In some embodiments, the method further includes treating the cardiovascular disease in the subject identified as having a cardiovascular disease or at increased risk of developing a cardiovascular disease. Methods of treating cardiovascular disease are well known in the art and a physician can select an appropriate therapy for a subject, depending in part of the particular type of cardiovascular disease to be treated.

### V. Methods of Measuring Total Fibrinogen and γ′ Fibrinogen Levels

Methods of measuring total fibrinogen concentration and γ′ fibrinogen concentration in a sample are well known in the art. Several exemplary procedures are described herein; however, these examples are not intended to be limiting. The methods disclosed herein of diagnosing a cardiovascular disease and determining a subject's risk for a cardiovascular disease can be carried out using any suitable method to measure total plasma fibrinogen and γ′ fibrinogen concentrations. Plasma samples are obtained using routine collection procedures.

A. Methods of Measuring Total Fibrinogen

A number of different total fibrinogen assays are well known in the art and routinely used in clinical laboratories (de Maat et al., “Fibrinogen” in Laboratory Techniques in Thrombosis: A Manual, J. Jespersen, R. M. Bertina & F. Haverkate eds., pp. 79-88, Kluwer Academic Publishers, Dordrecht).

In some embodiments, total fibrinogen concentration is measured according to the method described by Clauss (Acta Haematol 17:237-246, 1957), which is based on the thrombin clotting time. Mackie et al. (British Journal of Haematology 121:396-404, 2003) provide a summary of the Clauss method. Briefly, a high concentration of thrombin (generally about 100 U/ml) is added to dilute test plasma and the clotting time is measured. The result of the assay is compared with a calibration curve that is generated by clotting a series of dilutions of a reference plasma sample of known fibrinogen concentration, and a result in g/l is obtained.

Total fibrinogen concentration can also be determined by evaluating prothrombin time (PT-Fg). In this method, a calibration curve is generated using a sample with a known concentration of fibrinogen and plotting a graph of the optical change against fibrinogen concentration. The optical change in a test sample is converted to a fibrinogen concentration. This is an indirect measure of fibrinogen concentration (Mackie et al., British Journal of Haematology 121:396-404, 2003)

In other embodiments, total fibrinogen is measured using a clottable protein assay. Thrombin is added to plasma in the absence of calcium ions, the clot is washed and then dissolved in alkaline urea or other reagents, followed by a spectrophotometric protein assay or estimation (Gaffney and Wong, Thrombosis and Haemostasis 68:428-432, 1992; Mackie et al., British Journal of Haematology 121:396-404, 2003).

In yet other embodiments, total fibrinogen is measured using an immunological assay. For example, an ELISA can be performed using a monoclonal antibody specific for fibrinogen. Fibrinogen levels can also be determined with an immunoprecipitation test called functional intact fibrinogen (FiF). FiF uses a monoclonal antibody (45J) specific for the α-appendage on the intact fibrinogen molecule (Gargan et al., Coag Fibrinolysis 5:465-468, 1990).

B. Methods of Measuring γ′ Fibrinogen

Methods of measuring γ′ fibrinogen are described below in the Examples. An immunometric technique was developed to measure γ′ fibrinogen concentrations in plasma, and used to study the clinical utility of this test in samples from healthy individuals enrolled in the Framingham Offspring Study and in a separate case/control study of coronary artery disease (see Example 1). Monoclonal antibody 2.G2.H9, which is specific for the unique carboxyl terminal peptide of the fibrinogen γ′ chain, is used as a capture antibody. Sheep anti-human fibrinogen/horseradish peroxidase conjugate was used for detection, with 3,3′,5,5′-tetramethylbenzidine as the substrate. The linearity, imprecision, analytical specificity, and lower limit of quantification of the assay were evaluated. The reference interval for γ′ fibrinogen was determined in healthy individuals from the Framingham Offspring Study (n=2,879), and associations between γ′ fibrinogen and cardiovascular disease risk factors were quantified. The sensitivity and specificity of γ′ fibrinogen in evaluating CAD patients (n=133) was determined with receiver-operating characteristic (ROC) curve analysis.

In results obtained with these methods, the γ′ fibrinogen ELISA had within-run CVs of 13.4% at 0.127 g/L and 4.8% at 0.416 g/L. The limit of quantification at an imprecision of 20% was 0.10 g/L. The reference interval for healthy individuals was 0.088 to 0.551 g/L. ROC curve analysis of results from patients with coronary artery disease yielded an area under the curve of 0.76, with a diagnostic accuracy of 0.78 at a decision threshold of 0.30 g/L. Accordingly, γ′ fibrinogen shows excellent utility for cardiovascular risk analysis.

The γ′ fibrinogen levels were measured in 3,300 participants of the Framingham Heart Study Offspring Cohort. Associations of γ′ fibrinogen were examined using linear regression for continuous CVD risk factors and logistic regression for prevalent CVD and other dichotomous measures. There were significant associations of γ′ fibrinogen levels with age, sex, body mass index, smoking, diabetes, blood glucose, and triglycerides, and a significant inverse association with HDL cholesterol. γ′ fibrinogen was associated with prevalent CVD and myocardial infarction (multivariable-adjusted odds ratio 1.53 (95% CI 1.14-2.05) and 1.76 (95% CI 1.06-2.92), respectively, for the highest vs. lowest γ′ fibrinogen tertiles). γ′ fibrinogen was incompletely correlated with total fibrinogen. However, the adjusted odds ratio for subjects in both the highest tertile of γ′ fibrinogen and highest tertile of total fibrinogen was 2.17 (95% CI 1.42-3.32) and 3.08 (95% CI 1.41-6.72) for prevalent CVD and for prevalent myocardial infarction, respectively, compared with subjects in the lowest tertiles. Based on these results, γ′ fibrinogen is associated with CVD risk factors and prevalent CVD.

### EXAMPLES

### Example 1

### γ′ Fibrinogen—Evaluation of a New Assay for Study of Associations with Cardiovascular Disease

### Materials

Reagents were obtained from Fisher Scientific, Inc. unless otherwise specified. Monoclonal antibody 2.G2.H9, directed against the γ′ chain carboxyl terminus (available from Upstate, Inc.), was used for detection of γ′ fibrinogen. Plasminogen-free unfractionated human fibrinogen was obtained from Calbiochem, Inc. Standards for γ′ fibrinogen were prepared from plasma obtained from anonymous donors (see below).

### Framingham Offspring Study Plasma Samples

Plasma samples (3,300) were obtained from the Framingham Offspring Study (Kannel et al., Am J Epidemiol 110:281-290, 1979). These samples were collected during the seventh examination cycle (between 1998 and 2001) and were maintained at −70° C. until analysis. The samples from the Framingham Offspring Study were obtained from 2,879 individuals with no prior occurrence of cardiovascular disease, and 421 individuals with previously documented cardiovascular disease, as defined by the prior occurrence of myocardial infarction, coronary insufficiency, angina pectoris, stroke, transient ischemic attack, or intermittent claudication (Kannel et al., Am J Epidemiol 110:281-290, 1979). Descriptive statistics (mean±standard deviation for continuous risk factors, count and percent prevalence for dichotomous risk factors and γ′ and total fibrinogen) are presented. The significance of the mean/% difference across tertiles was assessed using age- and sex-adjusted analysis of covariance (continuous risk factors) or logistic regression (dichotomous risk factors).

### CAD Case/Control Samples

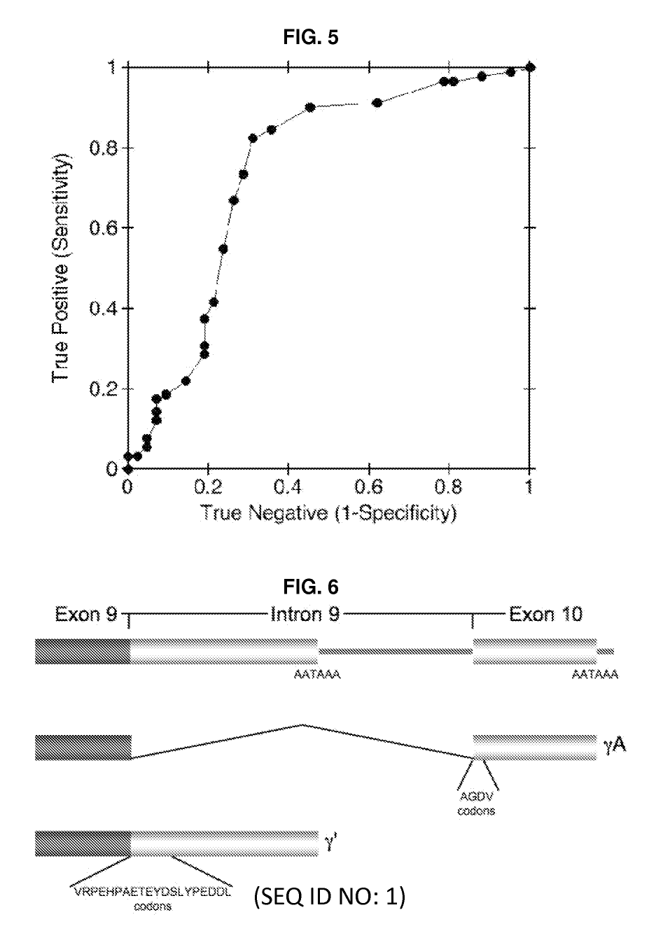

Data in FIG. 5 are based on a published case/control study of CAD (Lovely et al., Thromb Haemost 88:26-31, 2002). Briefly, blood was obtained from 133 patients between the ages of 41 and 80 who were referred for elective, outpatient diagnostic cardiac catheterizations. The indications for catheterization included anginal chest pain, positive stress test, valvular heart disease, and preoperative clearance prior to non-cardiac surgery in patients suspected of ischemic heart disease. Cases were defined as patients having luminal narrowing of 50% or greater in at least one major coronary artery or branch. Ninety-one cases of CAD were diagnosed, and 42 patients who had no angiographic evidence of disease were used as controls.

Data for γ′ fibrinogen concentrations measured in healthy controls showed a non-Gaussian distribution. Reference intervals were therefore established on the basis of the central 95^{th} percentile interval. The diagnostic performance of the test for differentiating patients with CAD from those without evidence of the disease was evaluated using Receiver Operating Characteristic (ROC) curve analysis (MedCalc Software). The sensitivity and specificity associated with incremental changes in γ′ fibrinogen concentrations were calculated and used to develop the ROC curve.

Preparation of γ′ Fibrinogen Standard

γA/γ′ fibrinogen was purified using a modification (Falls and Farrell, J Biol Chem 272:14251-14256, 1997) of a previous DEAE-cellulose chromatographic method (Mosesson et al., J Biol Chem 1247:5223-5227, 1972). To prepare the γ′ fibrinogen standard, normal human plasma (George King Biomedical) was first defibrinated by heating for 30 minutes at 56° C. followed by centrifugation for 30 minutes at 100,000×g to remove precipitated fibrinogen. Heat-defibrinated plasma was diluted 1:1000 in a solution of 0.1% fraction V BSA (Sigma) in PBS, which consisted of 0.137 mol/L NaCl, 2.7 mmol/L KCl, 10 mmol/L sodium phosphate, pH 7.4 (Sigma) containing 52 mmol/L EDTA and 0.1% Triton X-100 (v/v). Plasma samples were reconstituted to 1.5 μg/mL with DEAE cellulose-purified γA/γ′ fibrinogen. Serial dilutions of 0.75, 0.375, 0.188, 0.094, and 0.047 μg/mL were used in duplicate to calibrate the assay.

### Analytical Specificity of 2.G2.H9

Monoclonal antibody 2.G2.H9 was prepared in a bioreactor at the Penn State Hybridoma and Cell Culture Laboratory (State College, Pa.). To assess the specificity of the antibody for γ′ fibrinogen, 100 μl of 2.0 μg/mL γA/γA or γA/γ′ fibrinogen was coated on 96-well Maxisorp plates (Nunc) in 15 mmol/L Na2CO3, mmol/L NaHCO₃, pH 9.6, and blocked with 1% (w/v) BSA. Bioreactor supernatant was serially diluted in 120 mmol/L NaCl, 10 mmol/L Tris, pH 8.0/0.04% (v/v) Tween 20 and incubated for 1 hour at 37° C., followed by detection with goat anti-mouse IgG/HRP conjugate (Rockland Labs). One g/L O-phenylenediamine (Sigma) in 50 mmol/L sodium citrate, pH 4.5, 0.03% H₂O₂ was incubated at 22° C. until color development, which was read at 450 nm.

### γ′ ELISA

Monoclonal antibody 2.G2.H9 was purified as described previously (Lovely et al., Thromb Haemost 88:26-31, 2002). 96-well Maxisorp plates were coated overnight at 4° C. with 50 μl per well of a solution of 1.5 μg/mL 2.G2.H9 in PBS. Plates were then blocked for 1 hour at 37° C. with BSA in 250 μl PBS, 1% BSA and 0.1% Triton X-100. Citrated human plasma samples were diluted 1:1000 in PBS containing 5 mmol/L EDTA, 0.1% BSA and 0.1% Triton X-100, and 50 μl was added per well in triplicate and incubated for 1 hour at 37° C. Wells were washed three times with 250 μl of PBS containing 0.1% Triton X-100. HRP-conjugated sheep anti-human fibrinogen (Innovative Research, Inc.) was diluted 1:2500 in PBS containing 0.1% BSA and 0.1% Triton X-100, and 50 μwas added per well, incubating for 1 hour at 37° C. Wells were washed three times with 250 μl of PBS containing 0.1% Triton X-100. Substrate solution (50 μl). Super Sensitive 1 Component HRP Microwell Substrate (BioFX Laboratories) was added to each well and incubated 30 minutes at 22° C. The substrate reaction was terminated by adding 50 μl of stop solution, 450 nm liquid stop solution for TMB microwell (BioFX Laboratories), and the absorbance was read at 450 nm in a PowerWave XS microplate reader (Bio-Tek). Absorbance values of the standards were fitted to a non-linear equation for a second-degree polynomial by least squares error method with use of Kaleidagraph™ software (Synergy Software).

### Measuring Total Fibrinogen Concentration

Total fibrinogen concentration was measured according to the method disclosed in Clauss (Acta Haematol 17:237-246, 1957). The precision of the assay was determined as described in CLSI EP15 (User Demonstration of Performance for Precision and Trueness; Approved Guideline—Second Edition. CLSI document IP15-A2. Clinical and Laboratory Standards Institute, 940 West Valley Road, Suite 1400, Wayne, Pa. 19087-1898, USA, 2005). The lower limit of quantification of the assay was evaluated by analyzing eight separate pools of patient plasma that varied in γ′ fibrinogen concentrations from approximately 0.05 to 0.42 g/L.

### Analytical Specificity of 2.G2.H9 Towards γ′ Fibrinogen

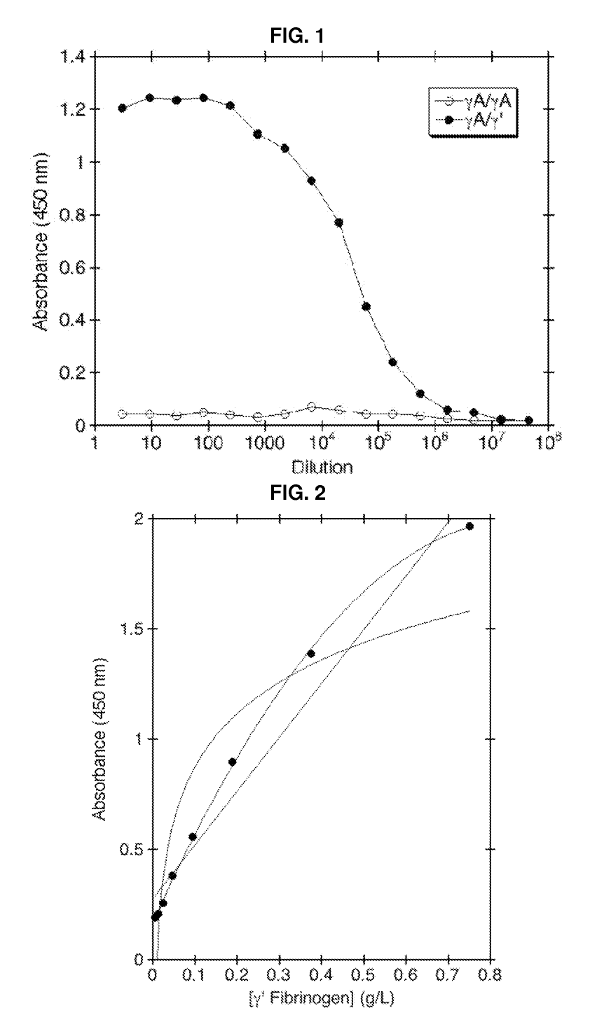

To assess the analytical specificity of anti-γ′ monoclonal antibody 2.G2.H9 and its ability to differentiate γ′ fibrinogen from γA/γA fibrinogen lacking γ′ chains, serial dilutions of antibody were reacted against purified γA/γA or γA/γ′ fibrinogen. As shown in FIG. 1, monoclonal antibody 2.G2.H9 showed no measurable reactivity towards γA/γA fibrinogen, even at the lowest dilutions. In addition, 2.G2.H9 showed only minor background reactivity with plasma that was heat-defibrinated to remove fibrinogen. Background absorbance with heat-defibrinated plasma was typically around 0.05 absorbance units. These results demonstrate the specificity of the antibody towards γ′ fibrinogen, which differs from γA/γA fibrinogen by only 20 out of 1,482 amino acids.

### γ′ Fibrinogen Standard Curve

A standard curve was generated using heat-defibrinated plasma that was reconstituted with purified γA/γ′ fibrinogen. The standard curve spanned the wide range of plasma concentrations of γ′ fibrinogen found in individuals, which can vary nearly 40-fold. This wide range of concentrations necessitated the use of non-linear curve fitting for the standard curve. FIG. 2 shows that on a representative standard curve, a linear curve fit yielded a fairly good approximation, with an R value of 0.980, whereas a logarithmic curve fit was clearly inferior, with an R value of 0.921. However, the linear curve fit showed significant skewing at the extremes of concentrations. A curve fit based on a second degree polynomial empirically provided the best fit, with an R value of 0.99 in this example. Importantly, this curve fit did not deviate at the extremes of concentrations as did the linear curve fit, which allowed all plasma samples to be assayed under the same conditions.

### Precision and Lower Limit of Quantification

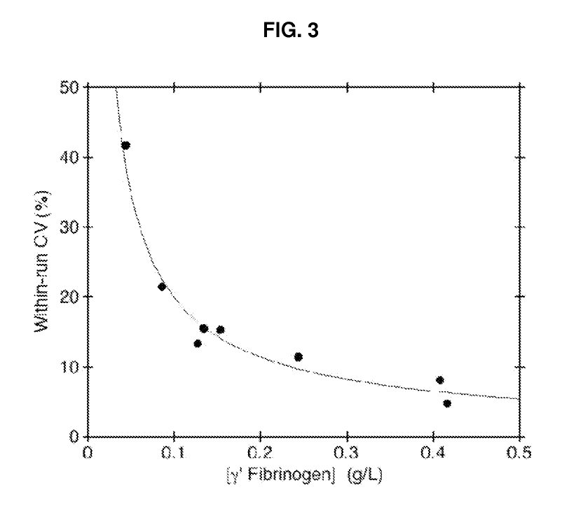

The precision of the method was evaluated by analyzing two separate pools of donor plasma with normal and increased γ′ fibrinogen concentrations. The pools were aliquotted into individual tubes, stored frozen at −70° C., and aliquots were analyzed, in duplicate, twice daily for 5 consecutive days. A plasma standard with a mean γ′ fibrinogen concentration of 0.127 g/L as established by 20 determinations had a within-run CV of 13.4% and a run-to-run CV of 28.6% with a total of 29.1%, whereas a plasma standard with a mean γ′ fibrinogen concentration of 0.416 g/L had a within-run CV of 4.8% and a run-to-run CV of 11.2% with a total of 11.6%. To determine the lower limit of quantification of the method, an aliquot from each of eight pools of donor plasma was measured in triplicate, three times a day, for three consecutive days. The mean γ′ fibrinogen concentration measured in each of the eight pools was plotted versus the within-run imprecision calculated for each pool. The lower limit of quantification was determined as that concentration of γ′ fibrinogen giving a within-run CV of 20%. FIG. 3 shows that the lower limit of quantification for this assay was 0.10 g/L.

### Distribution of γ′ Fibrinogen in Humans

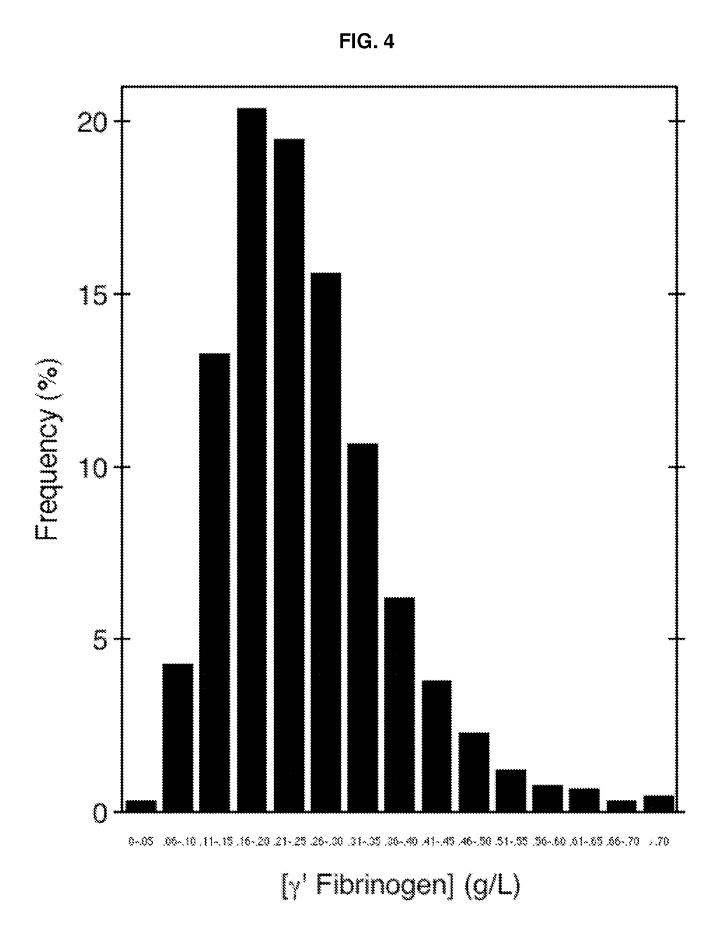

To establish the reference interval for γ′ fibrinogen in plasma, the concentration of γ′ fibrinogen was measured in plasma samples obtained from the seventh exam cycle (1998-2001) of the Framingham Offspring Study. γ′ fibrinogen was analyzed in participants with no previous history of cardiovascular disease (n=2,879). The characteristics of the entire Framingham Offspring cohort examined in this study are shown in Table 1. The distribution showed a substantial number of outliers with high concentrations of γ′ fibrinogen (FIG. 4). The range of γ′ fibrinogen measured in these samples varied nearly 40-fold, from a low of 0.037 g/L to a high of 1.443 g/L. The reference interval, defined as the 2.5^{th} and 97.5^{th} percentile limits for γ′ fibrinogen were 0.088 to 0.551 g/L, and the median concentration was 0.234 g/L. These median (2.5^{th} and 97.5^{th} percentiles) values were similar to those reported for 120 samples obtained from healthy blood donors, 0.281 g/L (0.115-0.460 g/L) (Lovely et al., Thromb Haemost 88:26-31, 2002) and from 42 healthy controls in a coronary artery disease case/control study (Lovely et al., Thromb Haemost 88:26-31, 2002) who showed a median γ′ fibrinogen of 0.242 g/L (0.125-0.676 g/L).

TABLE 1
Characteristics of the Framingham Offspring Cohort at Exam Cycle
(n = 3300)

| Variable | Value |
|---|---|
| Age (years) | 61 (±10) |
| Female (%) | 53.5 |
| Body mass index (kg/m²) | 28.2 (±5.3) |
| Cigarette smoking (%) | 13.0 |
| Diabetes mellitus (%) | 13.4 |
| Fasting blood glucose (mmol/L) | 5.79 (±1.52) |
| Systolic blood pressure (mmHg) | 127.0 (±18.8) |
| Total cholesterol (mmol/L) | 5.19 (±0.95) |
| HDL cholesterol (mmol/L) | 1.39 (±0.44) |
| Triglycerides (mmol/L) | 1.55 (±1.00) |
| γ′ fibrinogen (g/L) | 0.26 (±0.12) |
| Prevalent CVD (%) | 12.8 |

The association between γ′ fibrinogen and known cardiovascular disease risk factors was then examined in all the available samples from the seventh exam cycle (n=3,300). γ′ fibrinogen was significantly (all P<0.05) associated with age, sex, BMI, smoking, diabetes, blood glucose, and triglycerides (Table 2). Each of these risk factors increased significantly with increasing tertiles of γ′ fibrinogen. HDL cholesterol showed a statistically significant inverse association with tertiles of γ′ fibrinogen. Similar trends were seen in both men and women. In contrast to total fibrinogen concentrations (Kaptoge et al., Am J Epidemiol 166:867-879, 2007), γ′ fibrinogen did not show a significant association with systolic blood pressure or total cholesterol. These results suggest that γ′ fibrinogen is not simply a surrogate marker for total fibrinogen, but has different associations with known cardiovascular disease risk factors.

TABLE 2
Association of γ′ Fibrinogen with Traditional Cardiovascular Risk Factors.*

| | γ′ Fibrinogen Tertiles | | | |
|---|---|---|---|---|
| | Low | Mid | High | |
| | (0.03655-0.19827 mg/ml) | (0.19831-0.28564 mg/ml) | (0.28567-1.44290 mg/ml) | P- |
| Factor | (N = 1099) | (N = 1100) | (N = 1100) | Value^{†} |
| Age (years) | 58.8 (±9.0) | 60.9 (±9.6) | 63.5 (±9.3) | <0.001 |
| Female (%) | 50.3 | 54.7 | 55.5 | 0.023 |
| Body mass index (kg/m²) | 27.5 (±4.9) | 28.1 (±5.4) | 28.9 (±5.6) | <0.001 |
| Cigarette smoking (%) | 13.4% | 11.8% | 14.2% | 0.019 |
| Diabetes mellitus (%) | 10.3 | 11.8 | 17.8 | <0.001 |
| Fasting blood glucose (mmol/L) | 5.69 (±1.50) | 5.69 (±1.24) | 6.00 (±1.74) | <0.001 |
| Systolic blood pressure | 125.0 (±18.1) | 126.7 (±18.2) | 129.5 (±19.7) | 0.223 |
| (mmHg) | | | | |
| Total cholesterol (mmol/L) | 5.23 (±0.92) | 5.19 (±0.95) | 5.14 (±0.98) | 0.112 |
| HDL cholesterol (mmol/L) | 1.44 (±0.45) | 1.40 (±0.44) | 1.33 (±0.43) | <0.001 |
| Triglycerides (mmol/L) | 1.49 (±0.98) | 1.50 (±0.96) | 1.66 (±1.05) | <0.001 |
| Total fibrinogen (g/L) | 3.422 (±0.60) | 3.754 (±0.60) | 4.226 (±0.80) | <0.001 |
| γ′ fibrinogen (g/L) | 0.15 (±0.04) | 0.24 (±0.02) | 0.39 (±0.11) | — |

*Results are unadjusted mean (±1 standard deviation) or %
^{†}P-values assess the significance of the difference in mean/% across fibrinogen tertiles and are age and sex-adjusted (P-value for age is sex-adjusted only, and P-value for sex is age-adjusted only)

The ability of the γ′ fibrinogen assay to discriminate between individuals with CAD compared to controls without CAD was evaluated in order to calculate the optimum decision threshold (Zweig and Campbell, Clin Chem 39:561-577, 1993). This cohort (n=133) was investigated in a prior study (Lovely et al., Thromb Haemost 88:26-31, 2002). The mean age of this cohort was 62 (±10) years old, similar to the Framingham Offspring at cycle 7 (61 (±10) years old; see Table 1), although the CAD cohort was more predominantly male (57%). When γ′ fibrinogen concentrations were compared between CAD patients and non-CAD patients, γ′ fibrinogen concentrations were significantly higher in CAD patients (0.413±0.016 g/L vs. 0.299±0.024 g/L (mean 35 SE); P<0.0001), in both men and women. ROC curve analysis was used to evaluate the ability of γ′ fibrinogen concentrations to distinguish between patients with CAD and controls. The area under the ROC curve was 0.76 (FIG. 5). The point of the curve showing the optimum diagnostic accuracy was at a γ′ fibrinogen concentration of approximately 0.30 g/L. At this cutoff threshold the diagnostic accuracy was 0.78 for discriminating between patients with CAD and controls.

### Discussion

The ELISA method disclosed herein precisely measures levels of γ′ fibrinogen. A potential problem in the development of an ELISA for the quantitation of γ′ fibrinogen to avoid cross-reactivity towards the major fibrinogen isoform, γA/γA fibrinogen, which lacks γ′ chains. The results described herein show that monoclonal antibody 2.G2.H9 has no measurable cross-reactivity towards γA/γA fibrinogen. This finding is particularly important for epidemiologic studies since total fibrinogen, the vast majority of which is γA/γA fibrinogen, is already a well-established risk factor for cardiovascular disease (Danesh et al., JAMA 294:1799-1809, 2005). In addition, previous findings indicate that there is no significant association between γA/γA fibrinogen concentrations and γ′ fibrinogen concentrations in patients with CAD (Lovely et al., Thromb Haemost 88:26-31, 2002). These results suggest that γ′ fibrinogen is not simply a surrogate for γA/γA fibrinogen, but rather is an independent marker for CAD.

The skewed distribution of γ′ fibrinogen in the Framingham Offspring Study samples was unexpected, and was not seen previously in a smaller sampling of 120 plasma samples from Red Cross blood donors (Lovely et al., Thromb Haemost 88:26-31, 2002). This skewed distribution is not dissimilar from distributions of fibrinogen determined using the Clauss assay (Wang et al., N Engl J Med 355:2631-2639, 2006) as well as other circulating biomarkers, such as C-reactive protein (CRP). Although the Framingham Offspring Study participants in this analysis had no documented history of cardiovascular disease, the male and female participants were drawn from a general community dwelling population. It is likely that some of the participants may have had underlying subclinical cardiovascular disease that had not yet manifested itself as an acute event. ROC curve analysis showed a diagnostic accuracy of γ′ fibrinogen of 0.78 for discriminating patients with CAD, defined as >50% narrowing in at least one major coronary artery or branch, from individuals with <50% narrowing. This degree of accuracy was achieved with no adjustment for other variables such as age or gender.

Taken together, these studies indicate that γ′ fibrinogen can be used as a marker for cardiovascular disease. The addition of this marker to other established risk factors such as high-sensitivity CRP (Levinson and Will, Fats of Life Newsletter, Volume XVI No 2, Spring 2002) and cholesterol can provide additive predictive value for assessment of risk of adverse cardiac events.

### Example 2

### Association of γ′ Fibrinogen with Cardiovascular Disease—the Framingham Offspring Study

### Methods

### Study Population

The subjects in this study were from the Framingham Offspring Study. The design and methodology of this study for the long-term evaluation of risk factors for CVD have been described previously (Kannel et al., Am J Epidemiol 110:281-290, 1979). Plasma samples from the seventh examination cycle (1998-2001) from 3,300 individuals were assayed for γ′ fibrinogen.

### Determination of Risk Factors and Prevalent CVD

All visits preceding and including the seventh examination cycle included assessment of the prevalence of CVD and evaluation of CVD risk factors. New cases of myocardial infarction and stroke as well as related endpoints, such as angina, coronary insufficiency and transient ischemic attack, were adjudicated by a three-physician Endpoint Review Committee. Prevalent “hard CVD” events consisted of the prior occurrence of myocardial infarction or coronary insufficiency.

Prevalent “total CVD” consisted of the prior occurrence of myocardial infarction, coronary insufficiency, angina pectoris, transient ischemic attack, stroke, or intermittent claudication. Specific criteria for the clinical and laboratory methods and the CVD event adjudication have been published previously (Wilson et al., Circulation 97:1837-1847, 1998; Feinleib et al., Prey Med 4:518-525, 1975).

During the clinic visit at the seventh examination cycle, information was obtained on cigarette smoking during the past year and use of medications. Blood pressure after sitting for five minutes was measured using standardized methods. Phlebotomy took place under fasting conditions. Lipid determinations were made at the time of the examination in the Framingham Heart Study laboratory. Plasma cholesterol was measured according to the Lipid Research Clinics Program Protocol and high density lipoprotein (HDL) cholesterol was determined after precipitation of non-HDL lipoproteins with heparin-manganese (Manual of Laboratory Operations: Lipid Research Clinics Program, Lipid and Lipoprotein Analysis. 2nd ed. Washington, D.C.: National Institutes of Health, US Dept of Health and Human Services; 1982). Aliquots were frozen at −20° C. after the initial phlebotomy at the time of the baseline examination.

### γ′ Fibrinogen Analysis

γ′ fibrinogen was assayed using a modification of an ELISA described previously (Lovely et al., Thromb Haemost 88:26-31, 2002), and described in Example 1. 96-well Maxisorp plates were coated with 50 μl of 1.5 μg/ml monoclonal antibody 2.G2.H9 (Upstate USA, Inc., Charlottesville, Va.) in phosphate-buffered saline (PBS). The plates were blocked for 1 hour at 37° C. with bovine serum albumin (BSA) in 250 μl PBS/1% BSA/0.1% Triton X-100. Plasma samples were diluted 1:1,000 in PBS/5 mM EDTA/0.1% BSA/0.1% Triton X-100, and 50 μl was added in triplicate wells for 1 hour at 37° C. Wells were washed three times with 250 μl of PBS/0.1% Triton X-100. 50 μl of horseradish peroxidase (HRP)-conjugated sheep anti-human fibrinogen (Innovative Research, Inc., Southfield, Mich.) was diluted 1:2,500 in PBS/0.1% BSA/0.1% Triton X-100, and incubated in each well for 1 hour at 37° C. Wells were washed three times with 250 of PBS/0.1% Triton X-100. Fifty μl of TMB (3,3′,5,5′-tetramethylbenzidine) Super Sensitive 1 Component HRP Microwell Substrate (BioFX Laboratories) was added to each well and incubated for 30 minutes at 22° C. Fifty μl of 450 nm liquid stop solution for TMB microwell (BioFX Laboratories, Inc., Owings Mills, Md.) was added per well, and the absorbance was read at 450 nm in a PowerWave XS microplate reader (Bio-Tek). Absorbance values of the standards were fit to a non-linear equation for a second-degree polynomial with the Least Squared error method using Kaleidagraph™ software (Synergy Software, Reading, Pa.). The precision and variability of this assay has been independently validated. The ELISA coefficient of variability was 9.3% at the mean γ′ fibrinogen level.

### Statistical Analysis

Descriptive statistics (mean±standard deviation for continuous risk factors, count and percent prevalence for dichotomous risk factors and γ′ and total fibrinogen) are presented by γ′ fibrinogen tertile. The significance of the mean/% difference across tertiles was assessed using age- and sex-adjusted analysis of covariance (continuous risk factors) or logistic regression (dichotomous risk factors).

The significance of the difference in risk-factor adjusted mean γ′ fibrinogen between participants with and without prevalent disease was assessed using analysis of covariance. The significance of the distribution of each of prevalent CVD and myocardial infarction across γ′ fibrinogen tertiles was assessed using logistic regression adjusting for sex, age, body mass index (BMI), systolic blood pressure, diabetes mellitus, smoking, total cholesterol, HDL cholesterol and triglycerides. For each disease, the odds ratio (and its 95% confidence interval) relating the disease prevalence in the highest vs. lowest γ′ fibrinogen tertile is presented. An alpha of 0.05 was used to declare statistical significance.

### Results

### Association Between γ′ Fibrinogen and Traditional Cardiovascular Risk Factors

As shown in Table 3, there were statistically significant (all P<0.05) associations of γ′ fibrinogen levels with cardiovascular risk factors of age, sex, BMI, smoking, diabetes, blood glucose, and triglycerides. Each risk factor increased significantly with increased γ′ fibrinogen across tertiles. HDL cholesterol showed a statistically significant inverse association with γ′ fibrinogen. Similar trends were seen in both men and women. The tertiles for gamma′ fibrinogen determined in this study were <0.198 mg/ml, 0.198-0.286 mg/ml, and >0.286 mg/ml. The tertiles for total fibrinogen are <3.09 mg/ml, 3.09-3.68 mg/ml, and >3.68 mg/ml.

TABLE 3
Association of γ′ Fibrinogen with Traditional Cardiovascular Risk Factors.*

| | γ′ Fibrinogen Tertiles | | | |
|---|---|---|---|---|
| | Low | Mid | High | |
| | (0.03655-0.19827 mg/ml) | (0.19831-0.28564 mg/ml) | (0.28567-1.44290 mg/ml) | P- |
| Factor | (N = 1099) | (N = 1100) | (N = 1100) | Value^{†} |
| Age-years | 58.8 (±9.0) | 60.9 (±9.6) | 63.5 (±9.3) | <0.001 |
| % Female | 50.3% | 54.7% | 55.5% | 0.023 |
| Body mass index-kg/m² | 27.5 (±4.9) | 28.1 (±5.4) | 28.9 (±5.6) | <0.001 |
| Cigarette smoking-% | 13.4% | 11.8% | 14.2% | 0.019 |
| Diabetes mellitus-% | 10.3% | 11.8% | 17.8% | <0.001 |
| Fasting blood glucose-mg/dl | 102.5 (±27.1) | 102.6 (±22.4) | 108.1 (±31.4) | <0.001 |
| Systolic blood pressure-mmHg | 125.0 (±18.1) | 126.7 (±18.2) | 129.5 (±19.7) | 0.223 |
| Total cholesterol-mg/dl | 201.9 (±35.7) | 200.2 (±36.7) | 198.6 (±37.7) | 0.112 |
| HDL cholesterol-mg/dl | 55.6 (±17.4) | 53.9 (±16.9) | 51.5 (±16.6) | <0.001 |
| Triglycerides-mg/dl | 131.5 (±86.3) | 132.6 (±84.6) | 146.5 (±92.9) | <0.001 |
| γ′ fibrinogen-mg/ml¹ | 0.15 (±0.04) | 0.24 (±0.02) | 0.39 (±0.11) | — |
| Total fibrinogen-mg/dl | 342.2 (±60.0) | 375.4 (±60.3) | 422.6 (±80.1) | <0.001 |

*Results are unadjusted mean (±1 standard deviation) or %.
^{†}P-values assess the significance of the difference in mean/% across fibrinogen tertiles and are age and sex-adjusted (P-value for age is sex-adjusted only, and P-value for sex is age-adjusted only).
¹The tertiles for total fibrinogen are <3.09 mg/ml, 3.09-3.68 mg/ml, and >3.68 mg/ml.

### Association Between Prevalent CVD and γ′ Fibrinogen

Individuals with prevalent CVD had significantly higher risk-factor adjusted mean (±standard error) γ′ fibrinogen concentrations than those without CVD (0.278±0.006 mg/ml vs. 0.258±0.002 mg/ml; P=0.002). Results were similar for men and women separately (sex-by-prevalent CVD P-value=0.220). The age-adjusted odds ratio (95% confidence interval) comparing the prevalence of CVD in the highest vs. lowest γ′ fibrinogen tertile was 1.84 (1.15-2.93) for women, 1.70 (1.19-2.43) for men, and 1.76 (1.33-2.34) for men and women combined. This association (Table 4) remained significant after multivariable adjustment for sex, age, BMI, smoking, diabetes, fasting blood glucose, systolic blood pressure, total cholesterol, HDL cholesterol, and triglycerides for men and women combined [multivariable adjusted odds ratio 1.53 (1.14-2.05)]. Similarly elevated magnitudes of risk were noted in sex-specific analyses for women [multivariable adjusted odds ratio 1.66 (1.04-2.68)] and for men [multivariable adjusted odds ratio 1.44 (0.99-2.11)], although the relation was borderline significant in men.

TABLE 4
Association between prevalent cardiovascular disease and γ′ fibrinogen*

| | Adjusted Odds Ratio (95% CI) |
|---|---|
| Cardiovascular Disease | γ′ Fibrinogen Tertile 3 vs. 1 |
| Total CVD | 1.53 (1.14-2.05) |
| Hard CVD | 1.61 (1.05-2.47) |
| Myocardial infarction | 1.76 (1.06-2.92) |
| Stroke | 1.42 (0.68-2.95) |

*Both sexes combined; adjusted for sex, age, BMI, systolic blood pressure, fasting blood glucose, diabetes mellitus, smoking, total cholesterol, HDL cholesterol, and triglycerides.

Similar significant associations were found between γ′ fibrinogen and hard CVD [multivariable adjusted odds ratio 1.61 (1.05-2.47) for both sexes combined] and myocardial infarction [multivariable adjusted odds ratio 1.76 (1.06-2.92)]. The association between γ′ fibrinogen and stroke [multivariable adjusted odds ratio 1.42 (0.68-2.95)] did not reach statistical significance (P=0.36), although there was a non-significant trend towards higher γ′ levels with stroke. For all CVD outcomes, there was no significant γ′ fibrinogen-by-sex interaction on associations with prevalent CVD (P=0.082 for hard CVD, P=0.270 for myocardial infarction, P=0.335 for stroke).

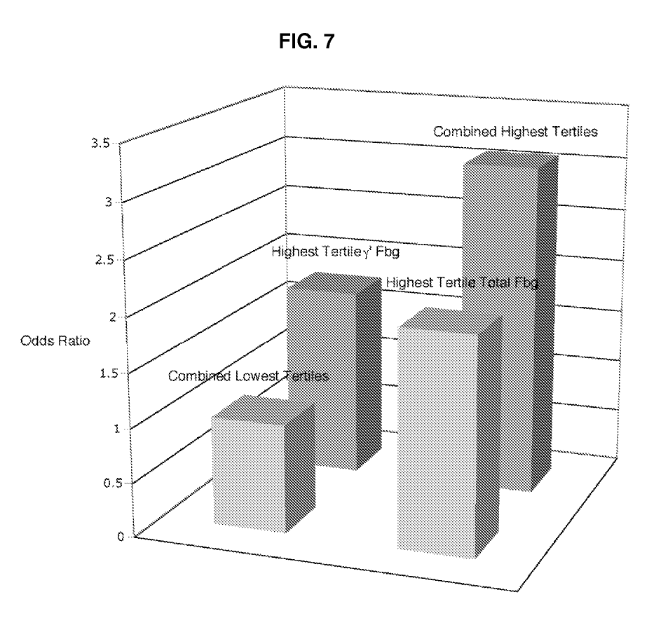

Further adjustment for total fibrinogen rendered insignificant associations of γ′ fibrinogen with CVD (P>0.05 for men and women combined). This is in part due to the very substantial correlation between the two fibrinogen measurements (age- and sex-adjusted Pearson correlation coefficient of 0.44; P<0.001). However, in multivariable-adjusted models, the odds ratio for the highest vs. lowest tertile of γ′ fibrinogen alone for CVD was 1.53 (1.14-2.05) and for the highest vs. lowest tertile of total fibrinogen alone, 1.54 (1.14-2.07), but the odds ratio for the highest tertile of both total fibrinogen and γ′ fibrinogen compared with the lowest tertile of both was 2.17 (1.42-3.32). The odds ratio for association between fibrinogen and myocardial infarction was also increased when considering both γ′ fibrinogen and total fibrinogen simultaneously than when considering either type of fibrinogen alone (FIG. 7). In multivariable-adjusted models, the odds ratio for highest vs. lowest tertile of γ′ fibrinogen alone was 1.76 (1.06-2.92) and for the highest vs. lowest tertile of total fibrinogen alone, 1.99 (1.21-3.28), but the odds ratio for the highest tertile versus the lowest tertile of both total fibrinogen and γ′ fibrinogen was 3.08 (1.41-6.72). These results suggest that γ′ and total fibrinogen are not simply surrogate markers for one another, but have different associations with cardiovascular disease.

### Genome-Wide Association Study

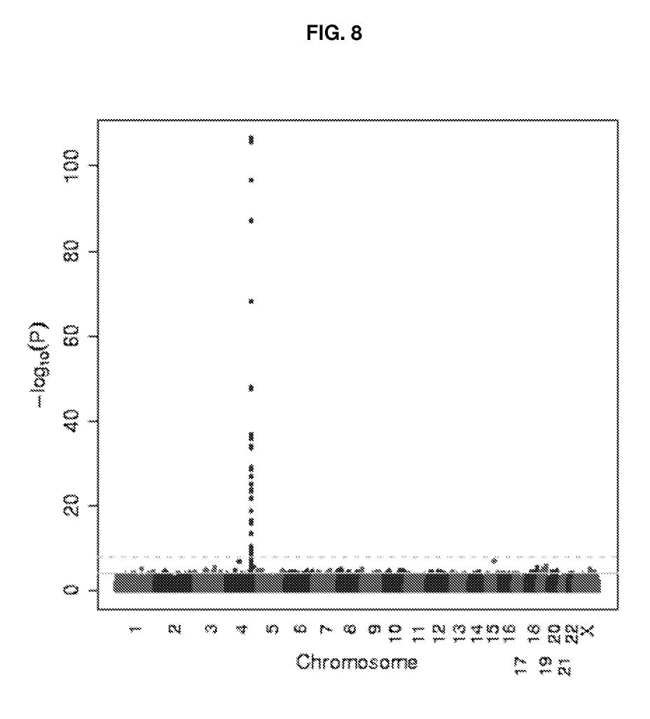

In order to identify genetic loci related to γ′ fibrinogen levels, a genome-wide association study was conducted. Quantile-quantile plots of the observed vs. expected P values showed little evidence of potential inflation in the results, with a lambda value of 1.01. A total of 46 single nucleotide polymorphisms (SNPs) exceeded the threshold for genome-wide significance (P<5.0×10⁻⁸) and clustered exclusively in or near the fibrinogen gene locus on chromosome 4 (FIG. 8). No other loci on other chromosomes reached the threshold for significance. The strongest statistical evidence for an association with γ′ fibrinogen levels was with rs7681423 (MAF: 0.225, P=2.25×10⁻¹⁰⁷), which is upstream of the 7 chain gene FGG (Table 5). Interestingly, the A>G variant in FGB with the strongest statistical association with total fibrinogen levels, rs1800789 (Dehghan et al., Circ Cardiovasc Genet 2:125-133, 2009), was not significantly associated with γ′ fibrinogen levels (MAF: 0.209, P=4.15×10⁻⁴).

TABLE 5
Identity of the Top SNPs with Significant Associations with
γ′ Fibrinogen Levels

| Locus | Location | Polymorphism | MAF | P Value |
|---|---|---|---|---|
| rs7681423 | 5′ of FGG | C > T | 0.225 | 2.25 × 10⁻¹⁰⁷ |
| rs7654093 | 5′ of FGG | A > T | 0.225 | 2.30 × 10⁻¹⁰⁷ |
| rs12644950 | 5′ of FGG | G > A | 0.225 | 2.63 × 10⁻¹⁰⁷ |
| rs2066861 | FGG intron 8 | C > T | 0.224 | 4.25 × 10⁻¹⁰⁷ |
| rs2066864 | FGG intron 9 | G > A | 0.225 | 1.14 × 10⁻¹⁰⁶ |
| rs2066865 | 3′ of FGG | G > A | 0.225 | 1.48 × 10⁻¹⁰⁶ |
| rs7659024 | between FGG | G > A | 0.225 | 2.33 × 10⁻¹⁰⁶ |
| | & FGA | | | |
| rs13130318 | 5′ of FGG | T > G | 0.217 | 1.90 × 10⁻⁹⁷ |
| rs13109457 | between FGG | G > A | 0.237 | 1.94 × 10⁻⁹⁷ |
| | & FGA | | | |
| rs6050 | FGA exon 5 | T > C | 0.239 | 6.82 × 10⁻⁸⁸ |
| | (Thr312Thr) | | | |
| rs6825454 | between FGA | T > C | 0.239 | 8.51 × 10⁻⁸⁸ |
| | & FGB | | | |
| rs6536024 | 5′ of FGG | C > T | 0.437 | 8.07 × 10⁻⁶⁹ |
| rs1049636 | FGG intron 9 | A > G | 0.323 | 1.16 × 10⁻⁴⁸ |
| rs1118823 | between FGG | T > A | 0.323 | 1.24 × 10⁻⁴⁸ |
| | & FGA | | | |
| rs12648395 | 5′ of FGG | T > C | 0.322 | 2.85 × 10⁻⁴⁸ |
| rs1800788 | 5′ of FGB | C > T | 0.194 | 1.49 × 10⁻³⁷ |
| rs12648258 | 5′ of FGB | T > A | 0.194 | 2.69 × 10⁻³⁷ |
| rs12642469 | 5′ of FGB | G > A | 0.192 | 1.14 × 10⁻³⁶ |
| rs13435101 | 3′ of PLRG1 | A > C | 0.474 | 9.68 × 10⁻³⁵ |
| rs12511469 | 3′ of PLRG1 | T > A | 0.192 | 2.01 × 10⁻³⁴ |
| rs13147579 | 3′ of PLRG1 | C > T | 0.192 | 2.40 × 10⁻³⁴ |
| rs7662567 | 3′ of PLRG1 | T > C | 0.192 | 2.41 × 10⁻³⁴ |
| rs10008078 | 3′ of PLRG1 | G > A | 0.192 | 2.42 × 10⁻³⁴ |
| rs12642770 | PLRG1 | T > C | 0.205 | 8.69 × 10⁻³⁰ |
| | intron 11 | | | |
| rs13435192 | 3′ of PLRG1 | T > C | 0.477 | 2.42 × 10⁻²⁹ |
| rs7689945 | 3′ of PLRG1 | T > C | 0.478 | 2.46 × 10⁻²⁹ |
| rs13123551 | 3′ of PLRG1 | A > T | 0.478 | 2.48 × 10⁻²⁹ |
| rs7659613 | 3′ of PLRG1 | G > C | 0.371 | 1.08 × 10⁻²⁷ |
| rs2070006 | between FGG | C > T | 0.371 | 1.39 × 10⁻²⁷ |
| | & FGA | | | |
| rs4463047 | between FGA | T > C | 0.0738 | 6.27 × 10⁻²⁶ |
| | & FGB | | | |
| rs4642230 | 3′ of PLRG1 | G > A | 0.174 | 1.32 × 10⁻²⁴ |
| rs2070011 | FGA promoter | C > T | 0.373 | 2.70 × 10⁻²⁴ |
| rs4235247 | 3′ of PLRG1 | G > A | 0.171 | 1.58 × 10⁻²² |
| rs2070018 | FGA intron 4 | A > G | 0.134 | 1.62 × 10⁻¹⁹ |
| rs4308349 | between FGA | A > G | 0.134 | 1.63 × 10⁻¹⁹ |
| | & FGB | | | |
| rs4550901 | between FGA | C > A | 0.134 | 1.63 × 10⁻¹⁹ |
| | & FGB | | | |
| rs12642646 | 3′ of PLRG1 | G > A | 0.460 | 2.49 × 10⁻¹⁷ |
| rs2070022 | FGA exon 6 | G > A | 0.178 | 1.05 × 10⁻¹⁶ |
| | (3′ UTR) | | | |
| rs2227412 | FGB intron 4 | A > G | 0.164 | 2.96 × 10⁻¹⁴ |
| rs9997519 | 5′ of PLRG1 | C > T | 0.165 | 3.19 × 10⁻¹⁴ |
| rs12651106 | DCHS2 | C > A | 0.174 | 3.37 × 10⁻¹¹ |
| | intron 2 | | | |
| rs4323084 | 3′ of PLRG1 | C > T | 0.230 | 2.53 × 10⁻¹⁰ |
| rs11737226 | 3′ of PLRG1 | A > G | 0.319 | 1.16 × 10⁻⁹ |
| rs6819508 | 3′ of PLRG1 | G > A | 0.146 | 2.52 × 10⁻⁹ |
| rs12645631 | PLRG1 | G > A | 0.147 | 2.73 × 10⁻⁹ |
| | intron 11 | | | |
| rs7698829 | PLRG1 | T > C | 0.147 | 2.77 × 10⁻⁹ |
| | intron 10 | | | |

Several SNPs were identified by genome-wide association that have previously been associated with γ′ fibrinogen levels, particularly rs1049636 (Mannila et al., J Thromb Haemost 5:766-773, 2007), the 9340C>T variant within intron 9 (MAF: 0.323, P=1.16×10⁻⁴⁸). In addition, rs2066861, the 7874G>A variant in intron 8 (MAF: 0.224, P=4.25×10⁻¹⁰⁷), rs 2066864, the 9615C>T variant in intron 9 (MAF: 0.225, P=1.14×10⁻¹⁰⁶), and rs2066865, the 10,034C>T variant in the 3′ untranslated region (MAF: 0.225, P=1.48×10⁻¹⁰⁶), which have previously been shown to be in linkage disequilibrium (Uitte de Willige et al., Blood 114:3994-4001, 2005), were associated with γ′ fibrinogen levels.

Perhaps the most intriguing association with SNPs near the fibrinogen gene locus was in the PLRG1 gene near the fibrinogen gene locus; rs12642770 (MAF: 0.205, P=8.69×10⁻³⁰), rs12645631 (MAF: 0.147, P=2.73×10⁻⁹), and rs7698829 (MAF: 0.147, P=2.77×10⁻⁹) in PLRG1 all showed genome-wide significance. PLRG1 encodes pleiotropic regulator 1, which plays a direct role in mRNA splicing. No SNPs in PLRG1 were found previously in association with total fibrinogen levels (Dehghan et al., Circ Cardiovasc Genet 2:125-133, 2009).

### Discussion

The results of the studies described above show a significant increased multivariable-adjusted association of plasma γ′ fibrinogen with prevalent total CVD as well as prevalent myocardial infarction. In addition, there is a higher order interaction between γ′ fibrinogen and total plasma fibrinogen that manifests as a further increased association with myocardial infarction. However, further adjustment for total fibrinogen attenuates the statistical significance of the association between γ′ fibrinogen and CVD. This is to be expected, since γ′ fibrinogen is a subset of total fibrinogen. By analogy, LDL cholesterol levels, a subset of total cholesterol, lose significant association with CVD if adjusted for total cholesterol levels. But in addition, variables that affect total fibrinogen expression may also affect γ′ fibrinogen expression. As one example, the FGG T9430C polymorphism in the fibrinogen 7 gene promoter, which increases total fibrinogen concentration, is also associated with increased plasma γ′ fibrinogen concentration (Mannila et al., J Thromb Haemost 5:766-773, 2007). So although there is significant correlation between total fibrinogen and γ′ fibrinogen, there is also considerable inter-individual variation between these two biomarkers. The partially additive effect of γ′ and total fibrinogen on the odds ratios for CVD indicates that γ′ and total fibrinogen are not simply surrogates for one another.

Another finding suggesting that γ′ and total fibrinogen are not surrogate markers comes from the genome-wide association study. The genetic loci associated with total fibrinogen levels (Dehghan et al., Circ Cardiovasc Genet 2:125-133, 2009) are different from those associated with γ′ fibrinogen levels in the current study. For total fibrinogen, four loci are marked by one or more single-nucleotide polymorphisms with genome-wide significance (P<5.0×10⁻⁸): FBG, the fibrinogen Bβ chain gene; IRF1, the interferon regulatory factor 1 gene; PCCB, the propionyl coenzyme A carboxylase gene; and NLRP3, the NLR family pyrin domain containing 3 isoforms gene. In contrast, the loci identified that are significantly associated with γ′ fibrinogen levels are all located within the fibrinogen gene locus, including the PLRG1 gene.

The top SNP in the fibrinogen gene cluster that was reported in association with total fibrinogen levels (Dehghan et al., Circ Cardiovasc Genet 2:125-133, 2009) was not associated with γ′ fibrinogen levels. The strongest statistical evidence for an association with total fibrinogen levels was with rs1800789, which is located in exon 7 of the fibrinogen Bβ chain gene FGB; the association of this SNP with γ′ fibrinogen levels did not even reach statistical significance (P=4.15×10⁻⁴). Rather, the top SNP in association with γ′ fibrinogen levels was rs7681423 upstream of the γ chain gene FGG. These findings suggest that γ′ fibrinogen and total fibrinogen levels are under differential genetic control, consistent with the differential odds ratios in CVD observed in Table 4.

### Example 3

### Association of γ′ Fibrinogen with Cardiovascular Disease: Analysis of CAD and Non-CAD Patients

In re-evaluating the data from a study of CAD and non-CAD patients, a significant correlation was found between levels of γ′ fibrinogen and total fibrinogen with increased odds of having CAD (Lovely et al., Thromb. Haemost 88:26-31, 2001). Re-analysis of the data from this 2002 study data shows that being in the top tertile of γ′ fibrinogen and total fibrinogen together gave an odds ratio of 76, with a p-value of <0.000. This determination was made using tertile cutoff values of 0-0.300 mg/ml, 0.300-0.413, and >0.413 for γ′ fibrinogen, and 0-3.73 mg/ml, 3.73-4.90, and >4.90 mg/ml for total fibrinogen.

### Example 4

### Action in Response to Test Values in the Top Tertile of γ′ Fibrinogen and Total Fibrinogen

The disclosed assay is used to identify subjects who are likely to have cardiovascular disease, or who are at increased risk of cardiovascular disease. Once a subject is identified as being in the top tertile of γ′ plasma fibrinogen and total plasma fibrinogen, appropriate clinical action can be taken. For example, if a subject has not already been diagnosed with cardiovascular disease a more comprehensive cardiovascular work-up is performed. Physical examination can detect cardiovascular disease, for example by the present of abnormal heart sounds, pedal edema and rales or hypertension. A cardiovascular diagnostic work-up can include tests such as an EKG to detect any rhythm or conduction abnormalities, a chest x-ray (CXR) to identify cardiomegaly that may be associated with heart failure, measurement of cardiac enzymes (such as creatinine kinase and troponin that may indicate recent cardiac muscle damage), cardiac imaging (for example with MRI or echocardiogram), cardiac stress testing, computed tomography angiography, Holter monitoring, or even more invasive diagnostic testing such as cardiac catheterization, electrophysiological examinations, and peripheral angiograms. If cardiovascular disease is identified, appropriate therapy can be advised or initiated such as prescribing a pharmaceutical interventions (such as anti-hypertensive or lipid-lowering medications) or lifestyle modifications (such as dietary or exercise regimes). In more extreme cases, surgical interventions such as coronary artery by-pass or peripheral vascular surgery may be performed.

1. A method of detecting a subject having a cardiovascular disease, or at increased risk of developing a cardiovascular disease, comprising:
measuring the concentration of γ′ fibrinogen and the concentration of total fibrinogen in a blood sample obtained from the subject;
comparing the measured concentration of γ′ fibrinogen and total fibrinogen to control ranges of γ′ fibrinogen and total fibrinogen in a population; and
determining which tertile the measured concentrations of γ′ fibrinogen and total fibrinogen fall into compared to the concentrations of γ′ fibrinogen and total fibrinogen in the population,
wherein the subject is identified as having a cardiovascular disease, or at increased risk of developing a cardiovascular disease, if the measured concentration of both γ′ fibrinogen and total fibrinogen are in the top tertile.

2. The method of claim 1, wherein the top tertile for γ′ fibrinogen is a concentration of at least 0.20 mg/ml.

3. The method of claim 1, wherein the top tertile for total fibrinogen is a concentration of at least 3.0 mg/ml.

4. The method of claim 1, wherein the top tertile for γ′ fibrinogen is a concentration of at least 0.286 mg/ml and the top tertile for total fibrinogen is a concentration of at least 3.68 mg/ml.

5. The method of claim 1, wherein γ′ fibrinogen and total fibrinogen are measured in plasma isolated from the blood sample.

6. A method of detecting a subject having a cardiovascular disease, or at increased risk of developing a cardiovascular disease, comprising measuring the concentration of γ′ fibrinogen and the concentration of total fibrinogen in a plasma sample obtained from the subject, wherein a γ′ fibrinogen concentration of at least 0.20 mg/ml, and a total fibrinogen concentration of at least 3.0 mg/ml identifies the subject as having a cardiovascular disease, or at increased risk of developing a cardiovascular disease.

7. The method of claim 6, wherein a γ′ fibrinogen concentration of at least 0.286 mg/ml and a total fibrinogen concentration of at least 3.68 mg/ml diagnoses the subject with a cardiovascular disease, or at increased risk of developing a cardiovascular disease.

8. The method of claim 1, wherein the cardiovascular disease is atherosclerosis, coronary artery disease, angina, thrombosis, ischemic heart disease, transient ischemic attack, coronary insufficiency, peripheral vascular disease, myocardial infarction, cerebrovascular disease, intermittent claudication, hypertension or elevated cholesterol.

9. The method of claim 8, wherein the cardiovascular disease is myocardial infarction or coronary artery disease.

10. (canceled)

11. The method of claim 1, further comprising obtaining the blood or plasma sample from the subject.

12. The method of claim 1, wherein measuring the concentration of γ′ fibrinogen comprises contacting the sample with an antibody that specifically binds to γ′ fibrinogen.

13. The method of claim 12, wherein the antibody is a monoclonal antibody.

14. The method of claim 13, wherein the monoclonal antibody is 2.G2.H9.

15. A method of detecting a subject having a cardiovascular disease, or at increased risk of developing a cardiovascular disease, comprising:
measuring the concentration of γ′ fibrinogen and the concentration of total fibrinogen in a plasma sample obtained from the subject;
comparing the measured concentration of γ′ fibrinogen and total fibrinogen to control ranges of plasma γ′ fibrinogen and total fibrinogen in a population; and
determining which tertile the measured concentrations of γ′ fibrinogen and total fibrinogen fall into compared to the concentrations of γ′ fibrinogen and total fibrinogen in the population,
wherein the subject is identified as having a cardiovascular disease, or at increased risk of developing a cardiovascular disease, if the measured concentration of both γ′ fibrinogen and total fibrinogen are in the top tertile.

16. The method of claim 1, further comprising reducing cardiovascular disease risk, or treating the cardiovascular disease, or both, in the subject identified as having a cardiovascular disease or at increased risk of developing a cardiovascular disease.

17. (canceled)

18. The method of claim 6, wherein the cardiovascular disease is atherosclerosis, coronary artery disease, angina, thrombosis, ischemic heart disease, transient ischemic attack, coronary insufficiency, peripheral vascular disease, myocardial infarction, cerebrovascular disease, intermittent claudication, hypertension or elevated cholesterol.

19. The method of claim 18, wherein the cardiovascular disease is myocardial infarction or coronary artery disease.

20. The method of claim 6, further comprising obtaining the blood or plasma sample from the subject.

21. The method of claim 6, wherein measuring the concentration of γ′ fibrinogen comprises contacting the sample with an antibody that specifically binds to γ′ fibrinogen.

22. The method of claim 6, further comprising reducing cardiovascular disease risk, or treating the cardiovascular disease, or both, in the subject identified as having a cardiovascular disease or at increased risk of developing a cardiovascular disease.
